(19) United States
(12) Patent Application Publication
(10) Pub. No.: US 2021/0369718 A1
(43) Pub. Date: Dec. 2, 2021

(54) COMPOSITIONS FOR MENTAL ALERTNESS AND METHODS OF MAKING AND USING THEREOF

(71) Applicant: Seattle Gummy Company, Seattle, WA (US)

(72) Inventors: Feng Wan, Issaquah, WA (US); William Brenden Carlson, Shoreline, WA (US); Henry W. Guo, Issaquah (CN)

(21) Appl. No.: 16/322,496

(22) PCT Filed: Aug. 4, 2017
(86) PCT No.: PCT/US17/45484

§ 371 Date: Feb. 1, 2019

Related U.S. Application Data
(60) Provisional application No. 62/371,146, filed on Aug. 4, 2016.
(60) Provisional application No. 62/371,136, filed on Aug. 4, 2016.
(60) Provisional application No. 62/371,120, filed on Aug. 4, 2016.

(51) Int. Cl. A61K 31/522; A61K 9/06; A61K 47/26; A61K 31/51; A61K 31/525; A61K 31/4415; A61K 31/714; A61K 31/455; A61K 31/197; A61K 31/519; A61K 31/4188; A61K 36/74; A61K 36/82; A61K 36/77; A61K 36/185; A61K 36/752; A61K 36/37; A61K 31/137; A61K 36/13; A61K 36/16; A61K 36/258; A61K 31/198; A61K 36/81; A61K 36/254; A61K 33/06; A61K 33/26; A61K 33/30; A61K 33/14; A61K 33/34; A61K 33/24; A61K 33/32; A61K 33/42; A61K 33/18; A61K 33/04; A61K 36/064; A61K 31/07; A61K 31/375; A61K 31/593; A61K 31/355; A61K 31/122; A61K 31/05; A61K 31/01; A61K 36/88; A61K 36/8962; A61K 36/87; A61K 31/047; A61K 36/45; A61K 36/815; A61K 35/10; A61K 31/706; A61K 31/7004; A61K 31/205; A61K 31/385; A61K 47/12; A61K 47/46; A61K 9/00; A23L 33/125; A23L 33/15; A23L 33/105; A23L 33/165

(52) U.S. Cl. CPC A61K 31/522; A23V 2002/00; A61K 47/26; A61K 31/51; A61K 31/525; A61K 31/4415; A61K 31/714; A61K 31/455; A61K 31/197; A61K 31/519; A61K 31/4188; A61K 36/74; A61K 36/82; A61K 36/77; A61K 36/185; A61K 36/752; A61K 36/37; A61K 31/137; A61K 36/13; A61K 36/16; A61K 36/258; A61K 31/198; A61K 36/81; A61K 36/254; A61K 33/06; A61K 33/26; A61K 33/30; A61K 33/14; A61K 33/34; A61K 33/24; A61K 33/32; A61K 33/42; A61K 33/18; A61K 33/04; A61K 36/064; A61K 31/07; A61K 31/375; A61K 31/593; A61K 31/355; A61K 31/122; A61K 31/05; A61K 31/01; A61K 36/88; A61K 36/8962; A61K 36/87; A61K 31/047; A61K 36/45; A61K 36/815; A61K 35/10; A61K 31/706; A61K 31/7004; A61K 31/205; A61K 31/385; A61K 47/12; A61K 47/46; A61K 9/0056; A23L 33/125; A23L 33/15; A23L 33/105; A23L 33/165; A61K 9/06

(57) ABSTRACT

The application provides nutraceutical compositions supporting mental alertness. In one embodiment, the nutraceutical composition comprises an active component and a gelling component in a sufficient amount to provide a cohesive gelled product. The active component comprises a carbohydrate composition, a vitamin B composition, a caffeinated composition and an anxiolytic composition, wherein the carbohydrate composition comprises a high glycemic index (GI) sugar having a GI not less than 70.

### CROSS-REFERENCES TO RELATED APPLICATIONS

This application claims the benefit of priority from, and hereby incorporates by reference the entire disclosure, co-pending U.S. Provisional Applications No. 62/371,120, filed Aug. 4, 2016; 62/371,136, filed Aug. 4, 2016; 62/371,146, filed Aug. 4, 2016.

### TECHNICAL FIELD

The application relate generally to nutraceutical composition in gelled or gummy formula, methods of administration of various gummy or gelled compositions for nutraceuticals, and kits comprising various gummy composition for nutraceuticals.

### BACKGROUND

In highly paced modern life, mental alertness is highly desired. Drivers on a long distance driving, students cramming for exams and term papers, workers on a night shift, engineers and scientists on a long project often rely on coffee for achieving mental alertness. Caffeine has a variety of pharmacological effects on organ systems and neural functions, though the level and duration of the effect varies among individuals. Caffeine stimulates the central nervous system, reaching its maximum effect between thirty and sixty minutes after absorption; this is accompanied by a temporary increase in metabolic function.

### SUMMARY

The application provides nutraceutical compositions supporting mental alertness. In one embodiment, the nutraceutical composition comprises an active component and a gelling component in a sufficient amount to provide a cohesive gelled product. The active component comprises a carbohydrate composition, a vitamin B composition, a caffeinated composition and an anxiolytic composition, wherein the carbohydrate composition comprises a high glycemic index (GI) sugar having a GI not less than 70.

In one embodiment, the active component further comprises a mineral composition, a vitamin composition, an antioxidant composition, or a mitochondria boosting composition.

In one embodiment, the high GI sugar comprises glucose, maltose, starch, glucose syrup, or a combination thereof.

In one embodiment, the Vitamin B composition comprises Vitamin B1 (thiamine), Vitamin B2 (riboflavin), niacin, vitamin B6, vitamin B12, folate, pantothenic acid, biotin, or derivatives thereof. In one embodiment, the vitamin B composition comprises niacin, Vitamin B2, Vitamin B6, and Vitamin B12.

In one embodiment, the caffeinated composition comprises caffeine, extract or powder of coffee, guarana, kola nut, mate (Ilex paraguariensis), green tea (Camellia sinensis), green coffee bean, Catha edulis (Khat), or a combination thereof. In one embodiment, the caffeinated composition comprises pure caffeine. In one embodiment, the caffeinated composition comprises pure caffeine and a herbal extract or powder containing caffeine.

In one embodiment, the anxiolytic composition comprises Ginkgo biloba, ginseng, L-theonine, theobromine, theophylline, flavanols, cocoa, flavonoids, extract, powder or derivative thereof. In one embodiment, the anxiolytic composition comprises Ginkgo biloba, ginseng, its extract or isolate thereof. In one embodiment, wherein the ginseng comprises Asian ginseng (panax ginseng), Siberian ginseng (Eleutherococcus senticosus), American ginseng (Panax quinquefolius), indian ginseng (Withania somnifera), or a combination thereof. In one embodiment, the herbal composition consists essentially panax ginseng. In one embodiment, the herbal composition consists essentially of Ashwagandha, Tribulus terrestris, and Mucuna pruriens.

In one embodiment, the mineral composition comprises salts or chelates of calcium, iron, zinc, magnesium, sodium, chloride, potassium, copper, molybdenum, manganese, phosphorus, iodine, nickel, or selenium, selenium yeast, or a combination thereof. In one embodiment, the chelates are amino acid chelates or peptide chelates. In one embodiment, the mineral composition comprises a hydrolyzed collagen chelated mineral including without limitation calcium, magnesium, iron, or Zinc.

In one embodiment, the vitamin composition comprises vitamin A, C, D, E, K or a combination thereof.

In one embodiment, the antioxidant composition comprises bioflavonoids, vitamin E, vitamin C, resveratrol, coenzyme Q10, quercetin, rutin, lycopene, L-glutathione, N-acetyl cysteine, phenolics, anthocyanins, flavonoids, anthracenes, carotenoids, zeaxanthin, astaxanthin, xanthin, pomegranate, Ginkgo biloba, green tea, garlic, grapeseed, blackberry, elderberry, cranberry, blueberry, saffron, Sangre de grado (dragon's blood), lyceum barbarum (Gouqizi), its extract, powder, or isolates thereof.

In one embodiment, the mitochondria boosting composition comprises acetyl L-carnitine, alpha-lipoic acid, coenzyme Q10 (CoQ10, or ubiquinone), Shilajit extract or powder, nicotinamide riboside (NR), vitamin B, vitamin D, omega-3 fatty acids, magnesium, D-ribose, or a derivative or combination thereof.

In one embodiment, the nutraceutical composition further comprises Vitamin D, creatine, or derivatives thereof.

In one embodiment, the gelling component comprises gelatin, starch, pectin, gellan gum, gum Arabic, carrageenans, guar, agar, alginate, locust bean gum, xanthan, or derivatives thereof. In one embodiment, the starch comprises corn starch, tapioca starch, potato starch, wheat starch, dextrins, maltodextrins, thin-boiling starch, high amylose corn starch, instant starches, or derivatives thereof. In one embodiment, the nutraceutical composition is essentially free of gelatin.

In one embodiment, the nutraceutical composition further comprises an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the composition may include sweeteners including, for example, sugar, glucose syrup, corn syrup, high fructose corn syrup, juice concentrate, or mixtures thereof. In one embodiment, the sweetener comprises erythritol, xylitol, sugar, glucose syrup, corn syrup, high fructose corn syrup, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl)propyl]-L-.alpha.-aspartyl]-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl)propyl]-L-alpha-aspartyl]-phenylal-anine 1-methyl ester, salts thereof, licorice or its extracts or isolates, or a mixture thereof.

The pH of the composition may be adjusted by the addition of suitable food acid, buffer, or both. The pH of the composition may be from about 3 to about 4.5, more preferably about 3.7 to about 4. In one embodiment, the buffer may be sodium or potassium tartrate.

In one embodiment, the food acid comprises malic acid, fumaric acid, lactic acid, tartaric acid, glucono-delta lactone, salts of gluconic acid, phosphoric acid, succinic acid, adipic acid, acetic acid, citric acid, or a combination thereof.

In one embodiment, the flavoring agent comprises vanilla, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, Rosemary Oil, lavender oil, sage oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, or mixtures or derivatives thereof.

In one embodiment, the bulking agent comprises maltitol syrup, polydextrose, sorbitol, soluble corn fiber, resistant starch, resistant maltodextrin, cellulose, hemicellulose, fructo-oligosaccharides, galacto-oligosaccharides, lactulose, xylo-isomalto-oligosaccharide, soybean oligosaccharide, oligo-glucose, stachyose, lactosucrose, or a combination thereof.

In one embodiment, the nutraceutical composition in this application has reduced sugar content when compared to conventional commercially available flavored gelled, chewy or gummy formulations. In one embodiment, when comparing to the conventional commercial products, the nutraceutical composition reduces sugar content by 25% to 75%. In one embodiment, the nutraceutical composition includes fructooligiosaccharides, a dietary fiber that has only 1.8 calories per gram, and thus lowers the calorie content of the composition. For example, the use of fructooligiosaccharides and water can provide a 20% to 30% reduction in calories, when compared to regular formulas with similar taste profiles.

In another aspect, the application provides methods for making the nutraceutical composition disclosed thereof. In one embodiment, the method includes the steps of: preparing a first premix by heating a mixture ingredients comprising a sweetener, a gelling component, and water sufficiently to dissolve in the water; preparing a gelling solution by heating a gelling composition, a vitamin B composition, a carbohydrate composition, a caffeinated composition and an anxiolytic composition a in water sufficiently to dissolve in the water; and blending the first premix and the gelling solution to form a combined mixture. In one embodiment, the method further comprises the steps of: mixing the Vitamin B composition, the carbohydrate composition, the caffeinated composition and the anxiolytic composition in presence of at least one gelling component to provide a mixture, and pulverizing the mixture until an average particle diameter of reaches 50 um or less.

The nutraceutical composition can be made in the typical manner or can be made utilizing an already made flavored gelled or gummy formulation as the base for the composition.

These and other objects and advantages of the application shall become apparent from the detailed description that follows.

### DETAILED DESCRIPTION

The application provides nutraceutical compositions for mental alertness. Example users for such composition include, without limitation, athletes, students, long distance drivers, and night shift workers.

In one embodiment, the nutraceutical composition includes a gelling component in a sufficient amount to provide a cohesive gelled product and an active component, wherein the active component consists of a vitamin B composition, a carbohydrate composition, a caffeinated composition, a mineral composition, an antioxidant composition, or an anxiolytic composition. In some embodiments, the nutraceutical composition may further include additives, including without limitation, sweeteners, food acids, flavoring agents, coloring agents, sensate agents, freshening agents, probiotics, prebiotics, bulking agents, hemectants, plasticizers, preservatives, stabilizing agents, emulsifiers, and thickening agents.

In some embodiments, the composition may include the active component from about 0% to about 25%, from about 0% to about 10%, from about 0% to about 5%, from about 1% to about 15%, from about 1% to 50%, from about 10% to about 60%, from about 1% to about 70%, by weight of the nutraceutical composition.

Carbohydrate Composition

The carbohydrate composition provides energy. In some embodiments, the carbohydrate composition may work synergistic with the caffeine composition to provide sustained energy and/or prolonger activity from caffeine.

Body uses glucose for energy. High glycemic index sugar such as glucose, maltose and starch quickly increase the blood glucose level after consumption and therefore leads to quick release of energy.

Maltose is a disaccharide consisting of two glucose units. The two glucose units are connected through a 1,4-alpha-glycosidic bond. Maltase is the enzyme that converts maltose into two units of glucose. The conversion of maltose into glucose is very rapid leading to rapid increase in glucose levels in the blood.

Blood glucose may work synergistically with caffeine from the caffeinated composition. For example, caffeine may increase local cerebral glucose consumption. The increase in circulating blood glucose leads to further increases in cerebral glucose consumption, thus magnifying the effects of caffeine in brain. In addition, caffeine may reduce the insulin sensitivity and therefore help maintain the blood glucose level.

Vitamin B Composition

B vitamins play an important role in athletic performance. The B-vitamins include thiamin, riboflavin, vitamin B-6, B-12 and folate, are necessary during the body's process for converting proteins and sugars into energy, and are used during the production and repair of cells, including red blood cells. The stress on the body's energy producing pathways during exercise, the changes in the body's tissues resulting from training, an increase in the loss of nutrients in sweat, urine and feces during and after strenuous activity and the additional nutrients needed to repair and maintain higher levels of lean tissue mass present in athletes and individuals with active life style may all affect an individuals B-vitamin requirements. Active individuals lacking in B-vitamins—including college athletes and other elite competitors—may perform worse during high-intensity exercise and have a decreased ability to repair and build muscle than counterparts with nutrient-rich diets.

In some embodiments, the composition includes a vitamin B composition. In some embodiments, the Vitamin B composition consists of folate, B6, and B12. In some embodiments, the vitamin B composition consists of thiamine (B1), riboflavin (B2), niacin (B3), panotothenic acid (B5), pyridoxine (B6), biotin (B7), folic acid (B9), cobalamins (B12), and derivatives or combinations thereof. In some embodiments, per serving dosing, the composition comprises at least 300 mcg, 400 mcg, 450 mcg, or 500 mcg of folate, at least 3 mg, 4.5 mg, 5.5 mg, 6.5 mg of B1, at least 3 mg, 5 mg, 6 mg, or 7 mg of B2, at least 12 mg, 15 mg, 16 mg, 18 mg, or 20 mg of niacinamide, at least 4 mg, 6 mg, 7 mg, 8 mg, 10 mg of B6, at least 15 mcg, 20 mcg, 30 mcg, 50 mcg, 1 mg, 2 mg, 4 mg, 5 mg, or 6 mg of B12, at least 30 mcg, 40 mcg, 50 mcg, 80 mcg, or 1 mg of biotin, or at least 7 mg, 10 mg, 12 mg, 15 mg, or 20 mg of pantothenic acid.

Caffeinated Composition

The caffeinated composition includes caffeine, extract or powder of coffee, guarana, kola nut, mate (Ilex paraguariensis), green tea (Camellia sinensis), green coffee bean, or a combination thereof. Any caffeine containing herbal extract or powder may be used. In one embodiment, the caffeinated composition includes caffeine. In one embodiment, the caffeinated composition includes extract or powder of guarana, green coffee bean extract or green tea. In one embodiment, the caffeinate composition may include caffeine and one or more of guarana, kola nut, mate (Ilex paraguariensis), green tea (Camellia sinensis), green coffee bean.

In one embodiment, the nutraceutical composition may include pure caffeine. In one embodiment, the nutraceutical composition may include a caffeine-containing herbal extract, powder or isolates thereof. In one embodiment, the nutraceutical composition may include pure caffeine and a caffeine-containing herbal extract, powder or isolates thereof.

In one embodiment, the nutraceutical composition may include, per serving, at least 5 mg, 10 mg, at least 20 mg, at least 30 mg, or at least 40 mg of pure caffeine or the total amount of pure caffeine and the caffeine from an herbal component.

Green tea is made from the leaves of the Camellia sinensis L plant, which is rich in polyphenol catechins and caffeine. Green tea provides health benefits for a wide variety of ailments, including the prevention of cancer and cardiovascular diseases, the anti-inflammatory, antiarthritic, antibacterial, antiangiogenic, antioxidative, antiviral, neuroprotecting, and cholesterol-lowering effects. The health-promoting effects of green tea are mainly attributed to its polyphenol content, particularly flavanols and flavonols, which represent 30% of fresh leaf dry weight. Recently, many of the aforementioned beneficial effects of green tea were attributed to its most abundant catechin, (−)-epigallocatechin-3-gallate (EGCG). Green tea extracts are more stable than pure epigallocatechin gallate, one of the major constituents of green tea, because of the presence of other antioxidant constituents in the extract.

Anxiolytic Composition

The anxiolytic composition may include agents that are capable of increasing cerebral blood flow, calming, or reducing anxiety or tiresomeness. The anxiolytic composition may counter act or neutralize caffeine's anxiety inducing effect. The anxiolytic composition may include Ginkgo biloba, ginseng, L-theonine, theobromine, theophylline, flavanols, cocoa, flavonoids, extract, powder or derivative thereof. The composition may include herbal extracts, isolates, powder or active phytochemicals.

In one embodiment, the anxiolytic composition comprises include Ginkgo biloba, ginseng, extract, power, or isolate thereof. In one embodiment, the anxiolytic composition comprises essentially ginseng, its extract or isolate thereof. In one embodiment, the anxiolytic composition comprises, per serving, not less than 5 mg, 10 mg, 15 mg, 20 mg, 30 mg of ginseng, its extract or isolate thereof. In one embodiment, the anxiolytic composition consists essentially of Ginkgo biloba. In one embodiment, the anxiolytic composition comprises, per serving, not less than 5 mg, 10 mg, 20 mg, 30 mg, 50 mg, 70 mg or 100 mg of Ginkgo biloba, its extract or isolate thereof. In one embodiment, the anxiolytic composition comprises L-theonine. In one embodiment, the anxiolytic composition consists essentially of Ginkgo biloba and L-theonine. In one embodiment, the anxiolytic composition comprises, per serving, not less than 5 mg, 10 mg, 20 mg, 30 mg, 50 mg, 70 mg or 100 mg of L-theonine.

Ginseng consists of several species belonging to the plant family Araliaceae. The two major forms are Chinese, Korean or Asian ginseng that belong to the genus Panax, and Siberian or Russian ginseng that belongs to the genus Eleutherococcus. The biologically active constituents in Panax ginseng are a complex mixture of triterpene saponins known as ginsenosides. Siberian, or Russian, ginseng consists of the dried roots and rhizome of Eleutherococcus senticosis, and contains phenolics, polysaccharides, and eleutherosides. In China, Eleutherococcus senticosus is known as wujiaseng or Ciwujia with active ingredients as ciwujianosides.

In some embodiments, the ginseng comprises Asian ginseng (panax ginseng), Siberian ginseng (Eleutherococcus senticosus), American ginseng (Panax quinquefolius), indian ginseng (Withania somnifera), or a combination thereof. In some embodiments, the herbal composition comprises essentially ginseng, or its extract or isolate thereof. In some embodiments, the herbal composition comprises per serving at least 5 mg, 10 mg, 20 mg, 30 mg, or 50 mg of ginseng extract or isolate thereof.

Ginkgo biloba (Gb) has demonstrated antioxidant and vasoactive properties as well as clinical benefits. Extract from Gb leaves has been used in traditional Chinese medicine for centuries to treat circulatory disorders, asthma, tinnitus, vertigo, and cognitive problems. Gb extract contains mainly terpenoids, flavonol glycosides, and proanthocyanidins. The most prevalent of these three groups are the flavonol glycosides (quercetin, catechin). The terpenoids include ginkgolides and bilobalides, which represent unique components of Gb. Terpenoids, flavonoids and proanthocyanidins are thought to be responsible for the pharmacological properties of Gb. Extract from Gb leaves inhibits platelet-activating factor and enhances NO production in vessels, with subsequent effect on peripheral and cerebral blood flow. Gb extract is thought to module different neurotransmitter systems: it is a strong inhibitor of monoamine oxidase A and synaptosomal uptake of DA, 5-HT, and norepinephrine. Additionally, Gb displays a free radical scavenger activity and has neuroprotecting and antiapoptotic properties, such as inhibition of amyloid-β neurotoxicity and protection against hypoxic challenges and increased oxidative stress.

Other Herbal Composition

Other herbal composition may be added. Examples herbal extracts and isolates include without limitation echinacea, spirulina, garlic, ginger, Leucojum aestivum, Ganoderma Lucidum, grapes, thermadrene, ma-huang, guarana, caffeine, purple willow bark, cayenne pepper, Kava kava (kava), St. Johns wort, Gamma oryzanol and Tribulus terrestris, Cordyceps sinensis, Rhodiola rosea, and Cytoseira canariensis. Example phytochemicals may include ferulic acid beta carotene, capsicanoids, anthocyanidins, bioflavinoids, d-limonene, isothiocyanates, cysteines, catechins and polyphenols, onions, phytosterols, isoflavones, lycopene, curcumin, caffeine, inositol hexanicotinate, glucosamine, chondroitin, msm, melatonin, serotonin, cartotenoids, chlorophyll, chlorophyllin, fiber, flavanoids, anthocyanins, cyaniding, delphinidin, malvidin, pelargonidin, peonidin, petunidin, flavanols, catechin, epicatechin, epigallocatechin, epigallocatechingallate (EGCG), theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, or combinations thereof.

In some embodiments, the herbal composition comprises schisandra, dang-gui, he-shou-wu, gotu kola (centella asiatica), ashwagandha, tribulus terrestris, mucunna pruriens, ciwujia, or extracts or isolates thereof. In some embodiments, the herbal composition consists of ginseng and Ganoderma Lucidum. In some embodiments, the herbal composition consists of ginseng and dang-gui, or its extract or isolate thereof.

Mineral Composition

In one embodiment, the mineral composition may include potassium, chromium pocolinate, magnesium and selenium. In one embodiment, the mineral composition may include ions of sodium, magnesium, chromium, iodine, iron, manganese, calcium, copper, fluoride, potassium, phosphorous, molybdenum, selenium, zinc, and combinations thereof. The minerals may be in the forms of salts or chelates.

Chromium pocolinate: Chromium(III) picolinate (CrPic3) is a bright-red coordination compound derived from chromium(III) and picolinic acid. Small quantities of chromium are needed for glucose utilization by insulin in normal health. Chromium has been identified to regulate insulin by increasing the sensitivity of the insulin receptor.

Selenium is a trace element. It is a constituent of more than two dozen selenoproteins that play critical roles in reproduction, thyroid hormone metabolism, DNA synthesis, and protection from oxidative damage and infection and therefore is nutritionally essential for humans.

Magnesium is involved in adenosine triphosphate (ATP) production from fatty acid oxidation, post-contractile muscular relaxation, and bone remineralization. It is also involved in phosphatidylglycerol (DPG) production, which is important to red blood cell formation. ATP, present in all cells but particularly in muscle cells, stores energy. Athletes lose magnesium through sweat and urine. In some embodiments, per serving dosage, the composition includes at least 400 mg, 500 mg, or 800 mg of magnesium.

Essential to antioxidant glutathione peroxidase (SeGPx) production, selenium is a free radical-scavenging tripeptide made up of glutamine, cysteine and glycine. It is concentrated in the lining of the GI tract and lungs, in the liver, and in skeletal muscle. Reducing muscular SeGPx increased cellular damage from prolonged exercise, supporting the theory that free radical-induced muscle damage causes muscle fatigue. In addition, selenium benefits athletes' immune function and helps repair cellular damage. In some embodiments, per serving dosage, the composition includes at least 150 mcg, 200 mcg, or 250 mcg of selenium.

Zinc aids in post-exertion tissue repair and in the conversion of food to fuel. Example zinc compounds include Zinc picolinate and monomethionate. In some embodiments, per serving dosage, the composition includes at least 25 mg, 30 mg, 50 mg, 60 mg, or 70 mg of Zinc.

In some embodiments, per serving dosage, the composition includes from at least 200 mg, 400 mg, 600 mg, 800 mg, 1000 mg, 1200 mg, or 1500 mg of calcium. In some embodiment, per serving dosage, the composition includes at least 2 mg, 5 mg, 10 mg, 15 mg, or 20 mg of iron.

Antioxidants

Exercise increases oxygen consumption and causes a disturbance of intracellular pro-oxidant-antioxidant homeostasis. In some embodiment, the nutraceutical composition includes an antioxidant composition.

In some embodiments, the antioxidant composition can include but are not limited to ascorbic acid, citric acid, rosemary oil, vitamin A, vitamin E, vitamin E phosphate, tocopherols, di-alpha-tocopheryl phosphate, tocotrienols, coenzyme Q10, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and combinations thereof. In some embodiments, the antioxidant composition may include extracts and active phytochemicals such as ferulic acid (from apples), ginseng, ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine, glucosamine, chondroitin, msm, melatonin, serotonin, and mixtures thereof.

In some embodiments, the antioxidant composition consists essentially of vitamin E, beta-carotene, and vitamin C. In some embodiments, per serving dosage, the composition includes at least 80 mg, 100 mg, 150 mg, 200 mg, 250 mg, 400 mg, or 500 mg of vitamin E. In some embodiments, per serving dosage, the composition includes at least 15 mg, 20 mg, 30 mg, or 50 mg of beta-carotene. In some embodiments, per serving dosage, the composition includes at least 500 mg, 600 mg, 800 mg, 1 g or 1.5 g of vitamin C.

Vitamin Composition

In some embodiments, vitamins can include fat-soluble vitamins such as vitamin A, vitamin D, vitamin E, and vitamin K and combinations thereof. In some embodiments, vitamins can include water-soluble vitamins such as vitamin C (ascorbic acid), the B vitamins, and choline.

Vitamin D receptor within the muscle may suggest a role for vitamin D in muscle tissue function. Adequate vitamin D intake reduces risk for conditions such as stress fracture, total body inflammation, infectious illness, and impaired muscle function. vitamin D status in athletes is variable and is dependent on outdoor training time (during peak sunlight), skin color, and geographic location. In some embodiments, per serving dosage, the composition includes at least 400 IU, 600 IU, 800 IU, 1000 IU, 1200 IU, 1500 IU, or 2000 IU of Vitamin D.

Mitochondria Boosting Composition

The mitochondria boosting composition may include acetyl L-carnitine, alpha-lipoic acid, coenzyme Q10 (CoQ10, or ubiquinone), Shilajit extract or powder, nicotinamide riboside (NR), vitamin B, vitamin D, omega-3 fatty acids, magnesium, D-ribose, or a derivative or combination thereof.

Energy Composition

The nutraceutical composition may further include an energy composition. In some embodiments, the energy composition may include creatine, lactic acid or salts thereof, L-arginyl-L(+)-lactate, or derivative thereof.

Fibers and Prebiotics

The composition may further include fibers or prebiotics. In one embodiment, the fibers may present in an amount of from about 0.001% to 80%, alternatively 0.001% to 5%, alternatively 5% to 10%, alternatively 10% to 15%, alternatively 15% to 40%, alternatively 40% to 60%, alternatively 60% to 80%, by weight of said composition. Any suitable fiber can be used. In one embodiment, a naturally derived fiber is used, including one or more selected from naturally derived inulin, inulin extract, synthetic inulin, hydrolysis products of inulin commonly known as fructooligosaccharides, galacto-oligosaccharides, xylooligosaccharides, oligo derivatives of starch, husks, brans, psyllium, polysaccharides, starches, polycarbophil, lignin, arabinogalactans, chitosans, oat fiber, soluble corn fiber, non-digestible corn dextrin, non-digestible wheat dextrin, locust bean gum and derivatives of locust bean gum, hydroxypropylmethyl cellulose (HPMC), pectin, and mixtures thereof.

In some embodiments, fibers may include inulin, wheat dextrin, or fructooligiosaccharides. Inulin, wheat dextrin, and fructooligiosaccharides may also act as a thickening agent and improve the texture of the composition. Various load rates of dietary fiber can be incorporated in the composition to create improved texture and at certain load rates can provide dietary benefits including promoting a healthy digestion system, controlling blood sugar levels, and providing probiotic benefits. The addition of the dietary fiber along with the remaining components allow for the addition of water that helps displace sugar within the flavored chewy or gummy confection.

Inulin is indigestible by human enzymes ptyalin and amylase, which are designed to digest starch. As a result, inulin passes through much of the digestive system intact. Inulin is a highly effective prebiotic, stimulating the growth of beneficial probiotic bacteria in the gut. Inulin is used in low fat products because of its ability to provide a creamy smooth texture to products. Inulin is a dietary fiber and is believed to activate beneficial good bacteria in the digestive tract. The activation of these bacteria is thought to reduce the risk of bowel cancer. Inulin has a mildly sweet taste, but does not affect blood sugar levels and is recommended for diabetics. Inulin has been clinically proven to increase calcium absorption. The inherent calcium in dairy foods is now an even better source of this bone-building mineral when inulin is added because inulin improves the body's uptake. People have used plants containing inulin to help relieve diabetes mellitus, a condition characterized by hyperglycemia and/or hyperinsulinemia. The embodiments can provide flavored chewy or gummy candy with inulin as the fiber component and calcium supplementation. This flavored chewy or gummy candy would have an improved absorption of calcium because of the inulin within its composition. Calcium is one of the top deficient minerals in the diet of most individuals, and products produced in accordance with the guidelines provided herein can provide great tasting flavored chewy or gummy confections that can help consumers get more calcium within their diets by eating our products.

In some embodiments, the composition may further include probiotics, Example probiotics include, but not limited to, lactic acid producing microorganisms such as Bacillus coagulans, Bacillus subtilis, Bacillus laterosporus, Bacillus laevolacticus, Sporolactobacillus inulinus, Lactobacillus acidophilus, Lactobacillus curvatus, Lactobacillus plantarum, Lactobacillus jenseni, Lactobacillus casei, Lactobacillus fermentum, Lactococcus lactis, Pedioccocus acidilacti, Pedioccocus pentosaceus, Pedioccocus urinae, Leuconostoc mesenteroides, Bacillus coagulans, Bacillus subtilis, Bacillus laterosporus, Bacillus laevolacticus, Sporolactobacillus inulinus and mixtures thereof. Breath fresheners are also known by the following trade names: Retsyn,™ Actizol,™ and Nutrazin.™ Examples of malodor-controlling compositions are also included in U.S. Pat. No. 5,300,305 to Stapler et al. and in U.S. Patent Application Publication Nos. 2003/0215417 and 2004/0081713 which are incorporated in their entirety herein by reference for all purposes.

Sweetener

In general, an effective amount of sweetener may be utilized to provide the level of sweetness desired, and this amount may vary with the sweetener selected. In some embodiments the amount of sweetener may be present in amounts from about 0.001% to about 3%, by weight of the composition, depending upon the sweetener or combination of sweeteners used. The exact range of amounts for each type of sweetener may be selected by those skilled in the art.

Sweeteners may include one or more monosaccharides or disaccharides. Examples include sugar, sucrose, invert sugar, dextrose, lactose, honey, malt syrup, malt syrup solids, maltose, fructose, granular fructose, maple syrup, rice syrup, rice syrup solids, sorghum syrup, refiners syrup, corn syrup, corn syrup solids, high fructose corn syrup, molasses, or combinations thereof.

In one embodiment, the sweetener include common sugars such as sucrose and glucose, polyols such as maltitol, erythritol, and isomalt, syrup sweeteners such as glucose syrup, corn syrup, high fructose corn syrup, and juice concentrates.

The amount of sweetener can range from 20 to 60%, 40 to 80% by weight or from 50 to 70% by weight based on the total weight of the composition. In one embodiment, the composition contains about 30 to about 80 wt %, or about 45 wt %, glucose syrup. The glucose syrup may be combined with about 0 to about 25 wt %, or about 19 wt %, water. In one embodiment, the composition may contain about 0 to about 50 wt %, or about 30 wt % common sugar. In one embodiment, the weight ratio of sucrose to corn syrup solids will be within the range of from about 1:2 to about 2:1, or from about 2:3 to 3:2. The sucrose can be employed in any suitable physical form to achieve the batching requirements. Typically, the sucrose will be granular and the corn syrup will preferably be added as a liquid having a DE of greater than 42, e.g., 63 DE. The higher DE values will decrease the tendency for granulation and aid in maintaining softness.

In one embodiment, artificial sweeteners can be used such as acesulfame K, aspartame, sucralose, d-tagatose, neotame, monatin, and acesulfame potassium (Ace-K), or combinations thereof.

The sweeteners involved may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:

(a) water-soluble sweetening agents such as dihydrochalcones, monellin, steviosides, lo han quo, lo han quo derivatives, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame), N-[N-(3,3-dimethylbutyl)-L-.alpha.-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro 1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideo-xy-beta-D-fructofuranoside, or 4,6,1′,6′-tetrachloro-4,6,1′,6′-tetradeoxygalacto-sucrose; and 4,6,1′,6′-tetradeoxy-sucrose, and mixtures thereof;

(e) protein based sweeteners such as thaumaoccous danielli (Thaumatin I and II) and talin; and

(f) the sweetener monatin (2-hydroxy-2-(indol-3-ylmethyl)-4-aminoglutaric acid) and its derivatives.

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In some embodiments, the sweetener may be a polyol. Polyols can include, but are not limited to glycerol, sorbitol, maltitol, maltitol syrup, mannitol, isomalt, erythritol, xylitol, hydrogenated starch hydrolysates, polyglycitol syrups, polyglycitol powders, lactitol, and combinations thereof.

In general, an effective amount of intense sweetener may be utilized to provide the level of sweetness desired, and this amount may vary with the sweetener selected. The intense sweetener may be present in amounts from about 0.001% to about 3%, by weight of the composition, depending upon the sweetener or combination of sweeteners used. The exact range of amounts for each type of sweetener may be selected by those skilled in the art.

Food Acids

The pH of the composition is about 3 to about 5, about 3.7 to about 4. The pH may be adjusted by a food acid, buffer, or both.

Suitable food acids include but are not limited to acetic acid, adipic acid, ascorbic acid, butyric acid, citric acid, formic acid, fumaric acid, glyconic acid, lactic acid, phosphoric acid, malic acid, oxalic acid, succinic acid, tartaric acid, or combinations thereof.

Suitable buffers include but are not limited to sodium citrate and potassium citrate. For example, an acid/buffer system is 1.33% of a 54% citric acid solution, buffered with sodium citrate.

The amount of acid will be in the typical range of from about 0.5 to about 2% by weight, e.g., about 1.25%. Higher acid (lower pH) results in a lack of structure while lower acid levels do not provide enough “acid bite” in the flavor profile.

Flavoring Agents

In some embodiments, the composition may further include a flavoring agent. Flavoring agents may include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof.

In some embodiments, the flavoring agents may include mint(s), menthol, menthone, isomenthone, camphor and eucalyptol, eucalyptol, camphor, borneol, fenchone, menthone and isomenthone, isopulegol, monomenthyl succinate, and menthyl lactate, menthone, isomenthone, borneol, fenchone, eucalyptus, ducalyptol, ethyl benzoate, neomenthol, d-fenchone, furfurylidene butyrate, bucchu fractions, sage oil, corn mint oil, rosemary, monomenthyl succinate, amyl salicylate, eugenol, phellendrene, propyl furoate, ethyl-3-hydroxy butyrate, hexyl valerate, anisyl propionate, anysyl butyrate, dihydrocarveol, or clary sag,

Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors whose release profiles can be managed include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below. In some embodiments, flavorants may chose from geraniol, linalool, nerol, nerolidal, citronellol, heliotropine, methyl cyclopentelone, ethyl vanillin, maltol, ethyl maltol, furaneol, alliaceous compounds, rose type compounds such as phenethanol, phenylacetic acid, nerol, linalyl esters, jasmine, sandlewood, patchouli, and/or cedarwood.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, e.g., melonal (melon), 2,6-dimethyloctanal (green fruit), and 2-dodecenal (citrus, mandarin), cherry, grape, blueberry, blackberry, strawberry shortcake, and mixtures thereof.

In some embodiments, flavoring agents are used at levels that provide a perceptible sensory experience i.e. at or above their threshold levels. In other embodiments, flavoring agents are used at levels below their threshold levels such that they do not provide an independent perceptible sensory experience. At subthreshold levels, the flavoring agents may provide an ancillary benefit such as flavor enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

Illustrations of the encapsulation of flavors as well as other additional components can be found in the examples provided herein. Typically, encapsulation of a component will result in a delay in the release of the predominant amount of the component during consumption of a composition that includes the encapsulated component (e.g., as part of a delivery system added as an ingredient to the chewing composition). In some embodiments, the release profile of the ingredient (e.g., the flavor, sweetener, etc.) can be managed by managing various characteristics of the ingredient, delivery system containing the ingredient, and/or the composition containing the delivery system and/or how the delivery system is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the composition, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc.

Sensate Agents

In some embodiment, the composition further includes a sensate agent. Sensate agents can include cooling agents, warming agents, tingling agents, effervescent agents, and combinations thereof. A variety of cooling agents may be employed. For example, among the useful cooling agents are included xylitol, erythritol, dextrose, sorbitol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N,2,3-trimethyl-2-isopropyl butanamide (WS-23), N-ethyl-p-menthane-3-carboxamide (WS-3), isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide, n-ethyl-t-2-c-6 nonadienamide, N,N-dimethyl menthyl succinamide, substituted p-menthanes, substituted p-menthane-carboxamides, 2-isopropanyl-5-methylcyclohexanol (from Hisamitsu Pharmaceuticals, hereinafter “isopregol”); menthone glycerol ketals (FEMA 3807, tradename FRESCOLAT® type MGA); 3-1-menthoxypropane-1,2-diol (from Takasago, FEMA 3784); and menthyl lactate; (from Haarman & Reimer, FEMA 3748, tradename FRESCOLAT® type ML), WS-30, WS-14, Eucalyptus extract (p-Mehtha-3,8-Diol), Menthol (its natural or synthetic derivatives), Menthol PG carbonate, Menthol EG carbonate, Menthol glyceryl ether, N-tertbutyl-p-menthane-3-carboxamide, P-menthane-3-carboxylic acid glycerol ester, Methyl-2-isopryl-bicyclo (2.2.1), Heptane-2-carboxamide; and Menthol methyl ether, and menthyl pyrrolidone carboxylate among others. These and other suitable cooling agents are further described in the following U.S. patents, all of which are incorporated in their entirety by reference hereto: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation can be provided. One such tingling sensation is provided by adding jambu, oleoresin, or spilanthol to some examples. In some embodiments, alkylamides extracted from materials such as jambu or sanshool can be included. Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of “tingling” type sensates can be found in U.S. Pat. No. 6,780,443, the entire contents of which are incorporated herein by reference for all purposes.

Sensate agents may also be referred to as “trigeminal stimulants” such as those disclosed in U.S. Patent Application No. 205/0202118, which is incorporated herein by reference. Trigeminal stimulants are defined as an orally consumed product or agent that stimulates the trigeminal nerve. Examples of cooling agents which are trigeminal stimulants include menthol, WS-3, N-substituted p-menthane carboxamide, acyclic carboxamides including WS-23, methyl succinate, menthone glycerol ketals, bulk sweeteners such as xylitol, erythritol, dextrose, and sorbitol, and combinations thereof. Trigeminal stimulants can also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin and menthol glycerin ether, unsaturated amides, and combinations thereof.

In some embodiments, sensate agents are used at levels that provide a perceptible sensory experience i.e. at or above their threshold levels. In other embodiments, sensate components are used at levels below their threshold levels such that they do not provide an independent perceptible sensory experience. At subthreshold levels, the sensates may provide an ancillary benefit such as flavor or sweetness enhancement or potentiation.

Freshening Agents

In some embodiments, the composition further includes a freshening agent. Freshening agents may include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments freshening agent can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof.

Coloring Agents

Coloring agents may be used in amounts effective to produce the desired color. The coloring agents may include pigments which may be incorporated in amounts up to about 6%, by weight of the composition. For example, titanium dioxide may be incorporated in amounts up to about 2%, and preferably less than about 1%, by weight of the composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-p-sulfoniumbenzylamino) diphenylmethylene]-[1-(N-ethyl-N-p-sulfoniumbenzyl)-delta-2,5-cyclohexadi-eneimine]. A full recitation of all F.D.& C. colorants and their corresponding chemical structures may be found in the Kirk-Othmer Encyclopedia of Chemical Technology, 3rd Edition, in volume 5 at pages 857-884, which text is incorporated herein by reference.

In some embodiments, one or more colors can be included. As classified by the United States Food, Drug, and Cosmetic Act (21 C.F.R. 73), colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), .beta.-apo-8′-carotenal (E160e), .beta.-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colors can include FD&C aluminum lakes. These include of the aluminum salts of FD&C dyes extended on an insoluble substrate of alumina hydrate. Additionally, in some embodiments, certified colors can be included as calcium salts.

In some embodiments, natural fruits or plant juice or extracts may be used as the coloring agents. Example include without limitation carrot juice, raspberry juice, blackberry juice, blueberry juice, and beet juice.

Plasticizer

In some embodiments, the composition may further include plasticizer to modify the texture of the formulation. A texture modifying agent may include a particulate material. Suitable particulate materials can include, but are not limited to, sucrose, polyols such as sorbitol, xylitol, mannitol, galactitol, lactitol, maltitol, erythritol, isomalt, hydrogenated starch hydrolysates and mixtures thereof, starches, proteins, and combinations thereof. In some embodiments, the particulate material serving as a texture modifying component is selected based on its ability or lack of ability to crystallize the saccharides in the saccharide portion. For example, when isomalt is included in the saccharide portion, sorbitol powder can be added to the composition because it will not cause the isomalt to crystallize. Alternatively, when erythritol is included in the saccharide portion, erythritol powder can be added to the composition because it will cause the erythritol to crystallize. Such particulates can be included in amounts from 5% to 35% w/w of the composition.

Fats and Oils

In some embodiments, a texture modifying component can include fats, oils, or other hydrophobic materials. Suitable fats can include, but are not limited to, partially hydrogenated vegetable or animal fats, such as coconut oil, corn oil, palm kernel oil, peanut oil, soy bean oil, sesame oil, cottonseed oil, cocoa butter, milk fat, beef tallow, and lard, among others. Suitable hydrophobic materials include chocolate, chocolate crumb, carob coatings, and compound coatings. Such fats, oils, and/or hydrophobic materials can be included in amounts of 1% to 10% w/w of the composition.

In some embodiments, the sensory perception of the texture modifying component is similar to that of fat, oil, or other hydrophobic materials. For example, a composition including 2.5% fats or oil can provide the same mouthfeel perception as a composition including 10%-50% fat as measured by sensory evaluation techniques.

Suitable oils and fats usable in compositions include vegetable or animal fats, such as butter, coconut oil, palm kernel oil, beef tallow, and lard, among others. These ingredients when used may be present in amounts up to about 7%, or up to about 3.5%, by weight of the composition.

In some embodiments, the composition may include edible oil component present in an amount of from about 1% to about 30%, alternatively 1% to 5%, alternatively 5% to 10%, alternatively 10% to 15%, alternatively 15% to 20%, alternatively 20% to 25%, alternatively 25% to 30%, by weight of the composition. In some embodiments, the edible oil component may be present in an amount of from about 0% to about 30%, alternatively 0% to 1%, alternatively 1% to 5%, alternatively 5% to 10%, alternatively 10% to 15%, alternatively 15% to 20%, alternatively 20% to 25%, alternatively 25% to 30%, by weight of the composition. This edible oil component makes up part of the group of ingredients that adjust the taste, texture, and improve the melt and mouth feel of the flavored chewy or gummy confection. For example, in some embodiments, the interaction of the group of highly unsaturated oils with the coconut oil component may create an improved elasticity within the flavored chewy confection that acts similar to hydrogenated or partially hydrogenated fat. The edible oil component also improves the health characteristic of the flavored chewy confection compositions because it adds monounsaturated and polyunsaturated fats. An example of an edible oil component is a blend of canola, soybean oil, and sunflower oil.

Non-limiting examples of edible oil components acceptable for use in the preferred embodiments include those that have low saturated fat content and high unsaturated fat including monounsaturated and especially polyunsaturated oils. The edible oil component should have no specific flavor and preferably is basically bland or somewhat buttery in taste. The edible oils component can be selected from the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof.

In some embodiment, the edible oil component may have a high level of saturated fats present in an amount of from about 0.3% to about 20%, alternatively 0.3% to 3%, alternatively 3% to 5%, alternatively 5% to 10%, alternatively 10% to 15%, alternatively 15% to 20%, by weight of the composition. Suitable oils having a high level of saturated fats include, but are not limited to, one or more selected from the group consisting of coconut oil, palm oil, fractionated coconut or palm oil, partially hydrogenated coconut or palm oil, fully hydrogenated coconut or palm oil, or any other synthesized or altered edible oils including partially hydrogenated oils and fully hydrogenated oils that have either highly saturated or highly unsaturated fatty acids that when hydrogenated become solid similar to coconut oil in consistency including partially hydrogenated soybean oil, cotton seed oil, palm kernel oil or combination of these edible oils. In one embodiment, the edible oil component comprises coconut oil. This oil component forms a part of the flavor profile and provides a texture to the flavored chewy or gummy confection, and it improves the taste, texture, melt, and mouth feel of the compositions. The blend of the flavor components provides for a great taste, texture, melt and mouthfeel, without the necessity of using partially hydrogenated or fully hydrogenated oils. Any medium heat processed coconut oil can be used.

Humectant

The glycerin is a humectant and freezing point depressant. It also helps decrease the tendency for granulation and aid in maintaining softness. In some embodiment, glycerin or equivalent material may be employed at a level of from about 1 to about 5% by weight of the final product, e.g., 2 to 3%.

Humectants that can provide a perception of mouth hydration may be included. Such humectants can include, but are not limited to glycerol, sorbitol, polyethylene glycol, erythritol, and xylitol. Additionally, in some embodiments, fats can provide a perception of mouth moistening. Such fats can include medium chain triglycerides, vegetable oils, fish oils, mineral oils, and combinations thereof.

Bulking Agents

Suitable sugar bulking agents include monosaccharides, disaccharides and polysaccharides such as xylose, ribulose, glucose (dextrose), lactose, mannose, galactose, fructose (levulose), sucrose (sugar), maltose, invert sugar, partially hydrolyzed starch and corn syrup solids, and mixtures thereof.

Suitable sugar alcohol bulking agents include sorbitol, xylitol, mannitol, galactitol, lactitol, maltitol, erythritol, isomalt and mixtures thereof. Suitable hydrogenated starch hydrolysates include those disclosed in U.S. Pat. No. 4,279,931 and various hydrogenated glucose syrups and/or powders which contain sorbitol, maltitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN®, a commercially available product manufactured by Roquette Freres of France, and HYSTAR®, a commercially available product manufactured by SPI Polyols, Inc. of New Castle, Del., are also useful.

Emulsifiers

The composition may include an emulsifier. The emulsifier may present in an amount of from about 0.001% to about 5%, alternatively 0.001% to 1%, alternatively 1% to 3%, alternatively 3% to 5%, by weight of the composition. In some embodiments, the emulsifier present in an amount of from about 0% to about 5%, alternatively 0.001% to 1%, alternatively 1% to 3%, alternatively 3% to 5%, by weight of the composition.

Example emulsifiers include but not limited to modified corn starch, mono- and diglycerides, and lecithin.

The emulsifier may assist in holding together the fats and water and other components together in a homogeneous composition. In one embodiment, the emulsifier may assist in the formation of a “water and oil” emulsion that creates the smooth texture of the finished product.

Preservatives

Preservatives may be natural or synthetic. Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof. Natural preservatives may include phenols (phenolic acid, polyphenols, tannins), isoflavonoids, organic acids (acetic, lactic, citric), and herb extracts such as extracts of citrus fruits, oregano, thyme, sage, rosemary, clove, coriander, garlic, and onion.

In some embodiments, the composition may include at least about 0% to 2%, by weight of the composition of a preservative component from above, or mixtures thereof.

Liquids

Liquids may be used to assist in the flavoring and texture profile of the products. In some embodiments, the composition may include from about 0.001% to about 25% by weight of a fruit or vegetable or combination juice or concentrate component, alternatively 0.001% to 5%, alternatively 5% to 10%, alternatively 10% to 15%, alternatively 15% to 20%, alternatively 20% to 25%, by weight of the composition. The fruit or vegetable or combination juice or concentrate component adds a flavor to the flavored chewy or gummy confection. Any suitable source from the following may used in the embodiments; citrus fruit juices, orchard fruit juices, berry fruit juices, vine fruit juices, decolorized juices, and vegetable juices can be used for this component. The forms can come from juices or concentrates of fruits or vegetables.

In some embodiments, the composition may also include a water component present in an amount of about 0% or greater. The water component adds to the overall texture and melt and chewiness of the flavored chewy or gummy composition. For example, water may be used because of the increase in viscosity of some example compositions. In some embodiments, the composition may contain water from about 1% to about 20%, alternatively 1% to 5%, alternatively 5% to 10%, alternatively 10% to 15%, alternatively 15% to 20%, by weight of the composition.

Thickening Agent

The composition may further include a thickening agent to help with the viscosity of the final product. Some thickening agents are gelling agents. Others act as mechanical thixotropic additives with discrete particles adhering or interlocking to resist strain.

In some embodiments, the thickening agent may be polysaccharides or protein. Example polysaccharides thickening agents include starches, vegetable gums and pectin. Example starch based thickening agents include arrowroot, cornstarch, katakuri starch, potato starch, sago, tapioca and their starch derivatives. Example vegetable gums based thickening agents may include alginin, guar gum, locust bean gum, and xanthan gum. Example protein based thickening agents include collagen, egg whites, furcellaran, and gelatin. Sugar based thickening agent may include agar and carrageenan.

Gelling Components

The gelling compounds may include one or more gelling agents. A number of gelling agents may be utilized including without limitation, gelatin, pectin, gum Arabic, carrageenans, agar agar, high methoxy pectin, alginates, xanthan gum, locust bean gum, gellan gum, guar gum, modified or unmodified starches, cellulose gum, modified starch wheat flour or enriched wheat flour or bleached flour or any type of flour from a natural source, or a combination thereof. Other example gelling agents may include acacia, alginic acid, bentonite, Carbopols® (now known as carbomers), carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, and tragacanth.

The amount of gelling agents used in the composition depend upon the texture, viscosity and softness of a desired product as well as other ingredients in the composition In some embodiments, to gelling agents may be used in concentrations of about 0.5% to about 10%, about 0.1% to about 7%, or about 0.2 to about 15%.

In one embodiment, the gelatin and pectin may be employed at a weight ratio supplying at least 50% gelatin and at least 10% pectin, e.g., from about 70 to 85% gelatin and the remainder pectin.

In one embodiment, pectin may be a high methoxy pectin obtained from apples. In one embodiment, gelatin may be a type A gelatin from porcine sources. Bloom values for the gelatin may be in the range of from 100 to 280. In one embodiment, the bloom value is about 250.

In one embodiment, the combination of gelatin and pectin may be employed at a level of from about 4.5 to about 6% by weight of the final product, e.g., about 5.5% on that basis.

In one embodiment, the composition may include gellan gum, carrageenans, or both providing a gelatin free composition. In one embodiment, the composition may include from about 0.25% to about 0.75% by weight gellan gum and about 2% to about 3% by weight carrageenan based on the total weight of the composition.

In one embodiment, a combination of gellan gum at about 0.25% to about 0.75% by weight and carrageenans at about 2.5% to about 3% by weight based on total weight of the product produces a gummy composition with TPA hardness values in excess of 20 lbs (f), and TPA cohesiveness and elasticity values of 75% to 80%.

In one embodiment, the amount of gellan gum is about 0.25 wt % to about 0.75 wt %, and about 0.25 wt % to about 0.5 wt %. In one embodiment, the amount of carrageenan is about 1.5 wt % to about 3 wt %, and about 2.5 wt % to about 3 wt %.

Texture Profile Analysis (TPA)

Texture Profile Analysis (TPA) is used to characterize the aspects of the texture of a gelled or gummy product. Specifically, TPA cohesiveness is a measure of the resiliency of a product, TPA elasticity is a measure of the springiness, and TPA hardness is a measure of firmness. For example, a gelatin gel tends to have high resiliency and high springiness, along with relatively high firmness.

Gelatin-based gummy composition may have both TPA cohesiveness and TPA elasticity values in the range of 80% to 90%, and TPA hardness values in the range of 25 to 30 lbs (f). Gelatin alternatives commonly found in the marketplace, such as starch and pectin, typically attain the proper TPA hardness, but have lower TPA elasticity values ranging from 30% to 50% and much lower TPA cohesiveness values ranging from 15% to 30%. Gellan gum has produced products with acceptable TPA hardness, TPA elasticity in the 50% to 70% range and TPA cohesiveness in the range of 30% to 50%. Carrageenan as a gelatin alternative have produced products with TPA cohesiveness and elasticity values of 75% and higher, but with unacceptably low TPA hardness values in the 10 to 15 lbs (f) range.

The following are typical TPA analysis of a variety of gelatin-based gummy products:

| | TPA Hardness | TPA | TPA |
|---|---|---|---|
| Product | lbs(f) | elasticity % | cohesiveness % |
| A | 30 +− 3 | 86 +− 1 | 82 +− 3 |
| B | 25 +− 2 | 91 +− 1 | 86 + 3 |
| C | 28 +− 2 | 90 +− 2 | 81 +− 1 |
| D | 28 +− 3 | 89 +− 3 | 83 +− 3 |

Methods of Making

Processing typically starts with the preparation of a boiled mixture of mixed sugars which is then blended with the gelling component and processed into shapes by depositing into starch molds. It can also be simply cast onto a slab or cast into rubber molds. The pieces are then held to set and dry. For a general description of this type of process, see Lees and Jackson; Sugar Confectionery and Chocolate Manufacture; 1973 (ISBN 0249 44120 9); pages 226-268. This reference is incorporated by reference herein in its entirety.

The method may further include a final processing process in which the final product is prepared. This process can include, for example, extruding, thermoforming, molding, shaping, cutting, and the like, to form the final product in the desired shape. Those skilled in the art are capable of designing a suitable final processing procedure to prepare the final gelled or gummy products, depending on the desired texture (e.g., chewy or gummy) and shape (e.g., cube, square, sheet, animal shaped, etc.).

The embodiments now will be described in more detail with reference to the non-limiting examples that follow.

### EXAMPLES

The following examples are provided to illustrate the preparation of gummy confections in accordance with this invention and are provided by way of illustration and are not intended to limit the invention in any way.

### Example 1. Caffeinated Gummy

Ingredients: 75 grams Citrus Pectin; Sodium Bicarbonate; Water; Glucose Syrup; Sucrose; Ginkgo; Green Coffee Bean Extract; Caffeine; 50% citric acid solution in water; Passion Fruit natural flavor

Water was added to a pan followed by sodium bicarbonate and caffeine. The sodium bicarbonate was allowed to dissolve and then the citrus pectin was added with stirring. The citrus pectin dissolved and the resulting solution was viscous, but free flowing, smooth and not granular.

The pectin solution was then heated and the glucose syrup was added. In a separate container, the sucrose, green coffee bean extract, and Ginkgo biloba extract were combined. The resulting mix was added to the pectin solution. The solution was heated and the citric acid solution was added followed by the addition of passion fruit natural flavor. The solution was then added to molds and allowed to cool to room temperature.

### Example 2: Caffeinated Gummy

Ingredients: Sodium Bicarbonate; Caffeine; Water; Citrus Pectin; Glucose Syrup; Sucrose; Green tea Extract; L-theonine; Apple Flavor; and Natural Coloring.

Combine water, caffeine, and sodium bicarbonate and bring to a boil. Cool the solution and add the pectin. Glucose syrup is then added.

In a separate container, the sucrose, green tea extract, and L-theonine are all combined. The resulting sugar mix is added to the pectin solution. The solution is heated and the citric acid solution is added with stirring followed by the addition of natural flavor. The solution is then added to molds and allowed to cool to room temperature.

### Example 3: Caffeinated Gummy

Ingredients: Sodium bicarbonate; Citrus Pectin; Water; Glucose Syrup; Sucrose; Caffeine; Green Coffee Bean Extract; 50% citric acid in water; B-Vitamin Mix; Ginseng Extract; Ginkgo biloba; and Natural flavor

Combine water caffeine, and sodium bicarbonate and allow the bicarbonate to dissolve, and add the pectin. Stir until homogenous and let stand for 15 minutes. Bring to a boil. Glucose syrup is then added.

In a separate container, the sucrose, ginseng, Gingko biloba and green coffee bean extract were all combined and the resulting sugar mix is slowly added to the pectin solution with stirring. The solution is heated and the citric acid solution was added followed by the addition of natural flavor.

The solution was then added to the molds and allowed to cool to room temperature. Within an hour the gelation occurred.

### Example 4: Caffeinated Gummy with Strawberry Flavor

Ingredients: Sodium Bicarbonate; Potassium Hydroxide food grade; Citrus Pectin; Water; VitaFiber Tapioca Syrup; Sucrose; Caffeine; 50% citric acid in water; B-Vitamin Mix; cocoa powder; Strawberry Extract; and Food Color

Combine water, caffeine, potassium hydroxide and sodium bicarbonate, and allow the bases to dissolve. Add the pectin. Stir until homogenous and let stand for 15 minutes. The tapioca syrup is then added.

In a separate container, the sucrose, cocoa powder are combined and shifted together until homogenous. The resulting sugar mix was added to the pectin solution with stirring. The citric acid solution was added with stirring followed by the addition of the B-vitamin premix, strawberry extract, and the color.

The solution was then added to silicone molds and allowed to cool to room temperature.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nutraceutical composition, comprising,
an active component, wherein the active component comprises a carbohydrate composition, a vitamin B composition, a caffeinated composition and an anxiolytic composition, wherein the carbohydrate composition comprises a high glycemic index (GI) sugar having a GI not less than 70, and
a gelling component in a sufficient amount to provide a cohesive gelled product.

2. The nutraceutical composition of claim 1, wherein the active component further comprises a mineral composition, a vitamin composition, an antioxidant composition, or a mitochondria boosting composition.

3. The nutraceutical composition of claim 1, wherein the high GI sugar comprises glucose, maltose, maltodextrine, starch, glucose syrup, or a combination thereof.

4. The nutraceutical composition of claim 1, wherein the Vitamin B composition comprises Vitamin B1 (thiamine), Vitamin B2 (riboflavin), niacin, vitamin B6, vitamin B12, folate, pantothenic acid, biotin, or derivatives thereof.

5. The nutraceutical composition of claim 1, wherein the vitamin B composition comprises niacin, Vitamin B2, Vitamin B6, and Vitamin B12.

6. The nutraceutical composition of claim 1, wherein the caffeinated composition comprises caffeine, extract or powder of coffee, guarana, kola nut, mate (Ilex paraguariensis), licorice, ephedra (Ephedra sinica or ma-huang), synepherine (Citrus aurantium), green tea (Camellia sinensis), green coffee bean, Catha edulis (Khat), or a combination thereof.

7. The nutraceutical composition of claim 1, wherein the anxiolytic composition comprises Ginkgo biloba, ginseng, L-theonine, theobromine, theophylline, flavanols, cocoa, flavonoids, extract, powder or derivative thereof.

8. The nutraceutical composition of claim 1, wherein the ginseng comprises Asian ginseng (Panax ginseng), Siberian ginseng (Eleutherococcus senticosus), American ginseng (Panax quinquefolius), indian ginseng (Withania somnifera), or a combination thereof.

9. The nutraceutical composition of claim 2, wherein the mineral composition comprises salts or chelates of calcium, iron, zinc, magnesium, sodium, chloride, potassium, copper, molybdenum, manganese, phosphorus, iodine, nickel, or selenium, selenium yeast, or a combination thereof.

10. The nutraceutical composition of claim 14, wherein the chelates are amino acid chelates or peptide chelates.

11. The nutraceutical composition of claim 2, wherein the vitamin composition comprises vitamin A, C, D, E, K or a combination thereof.

12. The nutraceutical composition of claim 2, wherein the antioxidant composition comprises bioflavonoids, vitamin E, vitamin C, resveratrol, coenzyme Q10, quercetin, rutin, lycopene, L-glutathione, N-acetyl cysteine, phenolics, anthocyanins, flavonoids, anthracenes, carotenoids, zeaxanthin, astaxanthin, xanthin, pomegranate, Ginkgo biloba, green tea, garlic, grapeseed, blackberry, elderberry, cranberry, blueberry, saffron, Sangre de grado (dragon's blood), lyceum barbarum (Gouqizi), its extract, powder, or isolates thereof.

13. The nutraceutical composition of claim 2, wherein the mitochondria boosting composition comprises acetyl L-carnitine, alpha-lipoic acid, coenzyme Q10 (CoQ10, or ubiquinone), Shilajit extract or powder, nicotinamide riboside (NR), vitamin B, vitamin D, omega-3 fatty acids, magnesium, D-ribose, or a derivative or combination thereof.

14. The nutraceutical composition of claim 1, wherein the gelling component comprises gelatin, starch, pectin, gellan gum, gum Arabic, carrageenans, guar, agar, alginate, locust bean gum, xanthan, or derivatives thereof.

15. (canceled)

16. The nutraceutical composition of claim 1, wherein the composition is essentially free of gelatin.

17. The nutraceutical composition of claim 1, further comprising an additive selected from sweeteners, food acids, flavoring agents, coloring agents, humectants, bulking agents, fatty acids, triglycerides, plasticizers, emulsifiers, thickeners, preservatives, or and a mixture thereof.

18. The nutraceutical composition of claim 17, wherein the sweetener comprises erythritol, xylitol, sugar, glucose syrup, corn syrup, high fructose corn syrup, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl)propyl]-L-.alpha.-aspartyl]-L-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

19. The nutraceutical composition of claim 17, wherein the food acid comprises malic acid, fumaric acid, lactic acid, tartaric acid, glucono-delta lactone, salts of gluconic acid, phosphoric acid, succinic acid, adipic acid, acetic acid, citric acid, or a combination thereof.

20. The nutraceutical composition of claim 17, wherein the flavoring agent comprises vanilla, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, Rosemary Oil, lavender oil, sage oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, or mixtures or derivatives thereof.

21. The nutraceutical composition of claim 17, wherein the bulking agent comprises maltitol syrup, polydextrose, sorbitol, soluble corn fiber, resistant starch, resistant maltodextrin, cellulose, hemicellulose, fructo-oligosaccharides, galacto-oligosaccharides, lactulose, xylo-isomalto-oligosaccharide, soybean oligosaccharide, oligo-glucose, stachyose, lactosucrose, or a combination thereof.

22. (canceled)

23. (canceled)

24. (canceled)
